(19) United States
(12) Patent Application Publication
(10) Pub. No.: US 2009/0306105 A1
(43) Pub. Date: Dec. 10, 2009

(54) Bicyclic heterocycles, medicaments containing these compounds, their use and process for preparing them

(75) Inventors: Frank Himmelsbach, Mittelbiberach (DE); Birgit Jung, Laupheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/281,190

(22) PCT Filed: Feb. 20, 2007
(86) PCT No.: PCT/EP2007/051589

§ 371 Date: Oct. 23, 2008

(30) Foreign Application Priority Data
Mar. 9, 2006 (EP) 06110886.6

(51) Int. Cl. A61K 31/517; C07D 239/72; A61P 11/00

(52) U.S. Cl. 514/266.24; 544/293

(57) ABSTRACT

The present invention relates to bicyclic heterocycles of the general formula (I), their tautomers, their stereoisomers, their mixtures and their salts, in particular physiologically tolerable salts with inorganic or organic acids, which have valuable pharmacological properties, in particular an inhibiting action on the signal transduction transmitted by tyrosinekinases, their use for the treatment of illnesses, in particular tumours and also benign prostatic hyperplasia (BPH), of disorders of the lungs and the bronchia and their preparation.

The present invention relates to bicyclic heterocycles of general formula

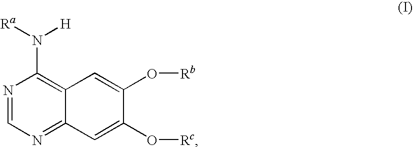

the tautomers, the stereoisomers, the mixtures and the salts thereof, in particular the physiologically acceptable salts thereof with inorganic or organic acids, which have valuable pharmacological properties, in particular an inhibitory action on signal transduction mediated by tyrosine kinases, their use for the treatment of illnesses, in particular of tumoral diseases and of benign prostatic hyperplasia (BPH), of diseases of the lung and of the airways, and the preparation thereof.

In the above general formula (I)

R^{a} denotes a 3-chloro-2-fluoro-phenyl, 3-chloro-4-fluoro-phenyl or 3-ethynylphenyl group,
R^{b} denotes a tetrahydrofuran-3-yl, tetrahydropyran-3-yl or tetrahydropyran-4-yl group, and
R^{c} denotes a tetrahydrofuran-3-yl, tetrahydropyran-3-yl or tetrahydropyran-4-yl group,
optionally in the form of the tautomers, the racemates, the enantiomers, the diastereomers and the mixtures thereof, and optionally the pharmacologically acceptable acid addition salts, solvates and hydrates thereof, preferably the tautomers, the racemates, the enantiomers, the diastereomers and the mixtures thereof, and optionally the pharmacologically acceptable acid addition salts thereof.

The compounds of general formula (I) may be prepared for example by the following methods:

a) reacting a compound of general formula

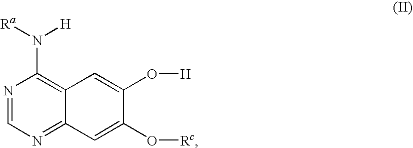

wherein
R^{a} and R^{c} are as hereinbefore defined, with a compound of general formula

Z¹-R^{b} (III),

wherein
R^{b} is as hereinbefore defined and Z¹ denotes a leaving group such as a halogen atom, e.g. A chlorine or bromine atom, a sulphonyloxy group such as a methanesulphonyloxy or p-toluenesulphonyloxy group or a hydroxy group.

The reaction is conveniently carried out in a solvent such as ethanol, isopropanol, acetonitrile, toluene, tetrahydrofuran, dioxane, dimethylformamide, dimethylsulphoxide or N-methylpyrrolidinone, optionally in the presence of a base such as potassium carbonate or N-ethyl-diisopropylamine, at temperatures in the range from 20° C. to 160° C., preferably at temperatures in the range from 40° C. to 120° C.

With a compound of general formula (III) wherein Z¹ denotes a hydroxy group, the reaction is carried out in the presence of a dehydrating agent, preferably in the presence of a phosphine and an azodicarboxylic acid derivative such as e.g. triphenylphosphine/diethyl azodicarboxylate, conveniently in a solvent such as methylene chloride, acetonitrile, tetrahydrofuran, dioxane, toluene or ethyleneglycol diethyl ether at temperatures in the range from −50 to 150° C., but preferably at temperatures in the range from 0 to 80° C.

b) reacting a compound of general formula

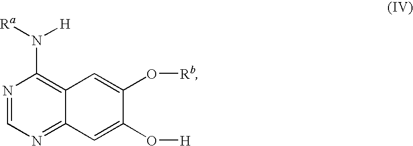

wherein
R^{a} and R^{b} are as hereinbefore defined, with a compound of general formula

Z¹-R^{c} (V),

wherein
R^{c} and Z¹ are as hereinbefore defined.

The reaction is conveniently carried out in a solvent such as ethanol, isopropanol, acetonitrile, toluene, tetrahydrofuran, dioxane, dimethylformamide, dimethylsulphoxide or N-methylpyrrolidinone, optionally in the presence of a base such as potassium carbonate or N-ethyl-diisopropylamine, at temperatures in the range from 20° C. to 160° C., preferably at temperatures in the range from 40° C. to 120° C.

With a compound of general formula (III) wherein Z¹ denotes a hydroxy group, the reaction is carried out in the presence of a dehydrating agent, preferably in the presence of a phosphine and an azodicarboxylic acid derivative such as e.g. triphenylphosphine/diethyl azodicarboxylate, conveniently in a solvent such as methylene chloride, acetonitrile, tetrahydrofuran, dioxane, toluene or ethyleneglycol diethyl ether at temperatures in the range from −50 to 150° C., but preferably at temperatures in the range from 0 to 80° C.

c) Reacting a compound of general formula

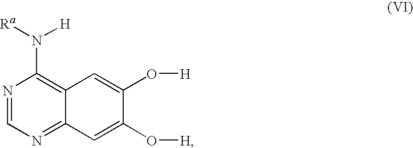

wherein
R^{a} is as hereinbefore defined, with a compound of general formula

Z¹-R^{d} (VI),

wherein
Z¹ is as hereinbefore defined and R^{d} denotes a tetrahydrofuran-3-yl, tetrahydropyran-3-yl or tetrahydropyran-4-yl group.

The reaction is conveniently carried out in a solvent such as ethanol, isopropanol, acetonitrile, toluene, tetrahydrofuran, dioxane, dimethylformamide, dimethylsulphoxide or N-methylpyrrolidinone, optionally in the presence of a base such as potassium carbonate or N-ethyl-diisopropylamine, at temperatures in the range from 20° C. to 160° C., preferably at temperatures in the range from 40° C. to 120° C.

With a compound of general formula (III) wherein Z¹ denotes a hydroxy group, the reaction is carried out in the presence of a dehydrating agent, preferably in the presence of a phosphine and an azodicarboxylic acid derivative such as e.g. triphenylphosphine/diethyl azodicarboxylate, conveniently in a solvent such as methylene chloride, acetonitrile, tetrahydrofuran, dioxane, toluene or ethyleneglycol diethyl ether at temperatures in the range from −50 to 150° C., but preferably at temperatures in the range from 0 to 80° C.

d) Reacting a compound of general formula (VIII)

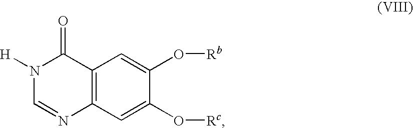

wherein R^{b} and R^{c} are as hereinbefore defined, with a halogenating agent, for example an acid halide such as thionyl chloride, thionyl bromide, phosphorus trichloride, phosphorus pentachloride or phosphorus oxychloride to form an intermediate compound of general formula (IX),

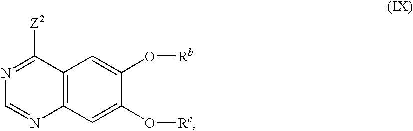

wherein R^{b} and R^{c} are as hereinbefore defined and Z² denotes a halogen atom such as a chlorine or bromine atom,
and subsequently reacting with a compound of general formula (X)

R^{a}—NH₂ (X),

wherein R^{a} is as hereinbefore defined.

The reaction with the halogenating agent is optionally carried out in a solvent such as methylene chloride, chloroform, acetonitrile or toluene and optionally in the presence of a base such as N,N-diethylaniline or N-ethyl-diisopropylamine at temperatures in the range from 20° C. to 160° C., preferably from 40° C. to 120° C. Preferably, however, the reaction is carried out with thionyl chloride and catalytic amounts of dimethylformamide at the boiling temperature of the reaction mixture, while the reaction may also be carried out with the addition of acetonitrile as solvent.

The reaction of the compound of general formula (IX) with a compound of general formula (X) is conveniently carried out in a solvent such as ethanol, isopropanol, acetonitrile, dioxane or dimethylformamide, optionally in the presence of a base such as potassium carbonate or N-ethyl-diisopropylamine, at temperatures in the range from 20° C. and 160° C., preferably from 60° C. to 120° C. Preferably, however, the reaction is carried out in isopropanol at the boiling temperature of the reaction mixture.

The compounds of general formula (I) obtained may be resolved into their diastereomers, for example by chromatographic methods.

Moreover the compounds of formula (I) obtained may be converted into their salts, particularly for pharmaceutical use into the physiologically acceptable salts thereof with inorganic or organic acids. Examples of acids include hydrochloric acid, hydrobromic acid, sulphuric acid, methanesulphonic acid, phosphoric acid, fumaric acid, succinic acid, lactic acid, citric acid, tartaric acid or maleic acid.

The compounds of general formulae (II) to (X) used as starting materials are known from the literature in some cases or may be obtained using methods known from the literature (cf. Examples I to VII), optionally with the additional inclusion of protective groups.

Compounds of general formulae II and IV may for example be obtained as described in WO 02/018351, WO 02/018372 or WO 03/082290.

The compounds of general formula I according to the invention and the physiologically acceptable salts thereof have valuable pharmacological properties, in particular an inhibitory action on the signal transduction mediated by the epidermal growth factor receptor (EGF-R), and this can be caused, for example, by an inhibition of ligand binding, receptor dimerisation or tyrosine kinase itself. Moreover, it is possible that the signal transmission to components lying further downstream is blocked.

The biological properties of the novel compounds were tested as follows:

The inhibition of human EGF receptor kinase was determined with the aid of the cyto-plasmic tyrosine kinase domain (methionine 664 to alanine 1186 based on the sequence published in Nature 309 (1984), 418). For this, the protein was expressed in Sf9 insect cells as a GST fusion protein using the Baculovirus expression system.

The measurement of the enzyme activity was carried out in serial dilutions in the presence or absence of the test compounds. The polymer pEY (4:1) from SIGMA was used as a substrate. Biotinylated pEY (bio-pEY) was added as a tracer/substrate. Each 100 μl of reaction solution contained 10 μl of the inhibitor in 50% DMSO, 20 μl of the substrate solution (200 mM HEPES pH 7.4, 50 mM magnesium acetate, 2.5 mg/ml poly(EY), 5 μg/ml bio-pEY) and 20 μl of enzyme preparation. The enzyme reaction was started by addition of 50 μl of a 100 μM ATP solution in 10 mM magnesium chloride. The dilution of the enzyme preparation was adjusted such that the phosphate incorporation into the bio-pEY was linear with respect to time and amount of enzyme. The enzyme preparation was diluted in 20 mM HEPES pH 7.4, 1 mM EDTA, 130 mM sodium chloride, 0.05% Triton X-100, 1 mM DTT and 10% glycerol.

The enzyme assays were carried out at room temperature over a period of 30 minutes and ended by addition of 50 μl of a stop solution (250 mM EDTA in 20 mM HEPES pH 7.4). 100 μl were transferred to a streptavidin-coated microtitre plate and incubated at room temperature for 60 minutes. The plate was then washed with 200 μl of a wash solution (50 mM tris, 0.05% Tween 20). After addition of 100 μl of an HRPO-labelled anti-PY antibody (PY20H Anti-PTyr:HRP from Transduction Laboratories, 250 ng/ml) the mixture was incubated for 60 minutes. The microtitre plate was then washed three times with 200 μl each of wash solution. The samples were then treated with 100 μl of a TMB-peroxidase solution (A:B=1:1, Kirkegaard Perry Laboratories). The reaction was stopped after 10 minutes. The extinction was measured at OD₄₅₀ₙₘ using an ELISA reader. All data points were determined as triplicates.

The data were fitted by means of an iterative calculation using an analysis program for sigmoidal curves (Graph Pad Prism Version 3.0). All analyses had a correlation coefficient of over 0.9. From the curves, the active compound concentration was derived which inhibits the activity of the EGF receptor kinase to 50% (IC₅₀). The compounds according to the invention have IC₅₀ values of less than 100 μm.

The compounds of the general formula I according to the invention thus inhibit the signal transduction by tyrosine kinases, as demonstrated using the example of the human EGF receptor, and are therefore useful for the treatment of pathophysiological processes which are caused by hyperfunction of tyrosine kinases. These are, for example, benign or malignant tumours, in particular tumours of epithelial and neuroepithelial origin, formation of metastases and the abnormal proliferation of vascular endothelial cells (neoangiogenesis).

The compounds according to the invention are also useful for the prevention and treatment of diseases of the airways and of the lung which are accompanied by increased or altered mucus production, which is caused by stimulation of tyrosine kinases, such as, for example, in inflammatory diseases of the airways such as acute bronchitis, chronic bronchitis, chronic obstructive bronchitis (COPD), asthma, bronchiectases, allergic or non-allergic rhinitis or sinusitis, nasal polyps, cystic fibrosis, α1-antitrypsin deficiency, or coughs, pulmonary emphysema, pulmonary fibrosis or hyperreactive airways.

The compounds are also suitable for treating inflammatory diseases of the gastrointestinal tract or bile duct and gall bladder which are associated with disrupted activity of the tyrosine kinases, such as may be found e.g. in acute or chronic inflammatory changes such as cholecystitis, Crohn's disease, ulcerative colitis, and ulcers or polyposis in the gastrointestinal tract or such as may occur in diseases of the gastrointestinal tract which are associated with increased secretions, such as Ménétrier's disease, secreting adenomas or protein loss syndromes,

and also for treating inflammatory diseases of the joints, such as rheumatoid arthritis, inflammatory diseases of the skin, the eyes, in inflammatory pseudopolyps, in colitis cystica profunda or pneumatosis cystoides intestinalis. The compounds may also be used for treating CNS and spinal cord injuries.

Preferred fields of application are inflammatory diseases of the respiratory organs or of the intestine, such as chronic bronchitis (COPD), chronic sinusitis, asthma, Crohn's disease, ulcerative colitis or polyposis of the intestines.

Particularly preferred fields of application are inflammatory diseases of the airways or lungs such as chronic bronchitis (COPD) or asthma or nasal polyps.

Moreover, the compounds of general formula (I) and the physiologically acceptable salts thereof can be used for the treatment of other diseases caused by aberrant function of tyrosine kinases, such as, for example epidermal hyperproliferation (psoriasis), benign prostatic hyperplasia (BPH), inflammatory processes, diseases of the immune system, hyperproliferation in haematopoietic cells, etc.

On account of their biological properties, the compounds according to the invention can be used alone or in combination with other pharmacologically active compounds, for example in tumour therapy in monotherapy or in combination with other antitumour therapeutics, for example in combination with topoisomerase inhibitors (e.g. etoposide), mitosis inhibitors (e.g. vinblastine), compounds interacting with nucleic acids (e.g. cis-platin, cyclophosphamide, adriamycin), hormone antagonists (e.g. tamoxifen), inhibitors of metabolic processes (e.g. 5-FU etc.), cytokines (e.g. Interferons), antibodies etc. For the treatment of airway diseases, these compounds can be used alone or in combination with other airway therapeutics, such as, for example, compounds having secretolytic activity (e.g. ambroxol, N-acetylcysteine), broncholytic activity (e.g. tiotropium or ipratropium or fenoterol, salmeterol, salbutamol) and/or anti-inflammatory activity (e.g. theophylline or glucocorticoids). For the treatment of diseases in the region of the gastrointestinal tract, these compounds can likewise be given alone or in combination with motility- or secretion-influencing substances. These combinations can be administered either simultaneously or sequentially.

The administration of these compounds either alone or in combination with other active compounds can be carried out intravenously, subcutaneously, intramuscularly, intraperitoneally, intranasally, by inhalation or transdermally or orally, aerosol formulations being particularly suitable for inhalation.

In the case of pharmaceutical administration, the compounds according to the invention are generally used in warm-blooded vertebrates, in particular in man, in doses of 0.01-100 mg/kg of body weight, preferably at 0.1-15 mg/kg. For administration, these are formulated with one or more conventional inert carriers and/or diluents, e.g. with maize starch, lactose, sucrose, microcrystalline cellulose, magnesium stearate, polyvinylpyrrolidone, citric acid, tartaric acid, water, water/ethanol, water/glycerol, water/sorbitol, water/polyethylene glycol, propylene glycol, stearyl alcohol, carboxymethylcellulose or fat-containing substances such as hard fat or suitable mixtures thereof, to form customary galenic preparations, such as tablets, coated tablets, capsules, powders, suspensions, solutions, sprays or suppositories.

The following examples are intended to illustrate the present invention in greater detail without restricting it:

### PREPARATION OF THE STARTING COMPOUNDS

### Example I

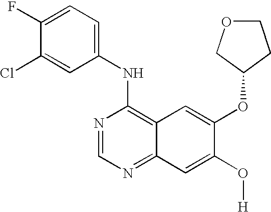

### 4-[(3-chloro-4-fluoro-phenyl)amino]-6-((S)-tetrahydrofuran-3-yloxy)-7-hydroxy-quinazoline

### a) benzyl 2-nitro-4-benzyloxy-5-((S)-tetrahydrofuran-3-yloxy)-benzoate

Prepared by reacting 6-nitro-benzo[1,3]dioxole-5-carboxylic acid with (S)-3-hydroxy-tetrahydrofuran in the presence of potassium-tert.-butoxide and subsequent reaction of the reaction mixture with benzyl bromide.

cf. Example VI (2), WO 03/082290

Mass spectrum (ESI⁺): m/z=450 [M+H]⁺

### b) benzyl 2-amino-4-benzyloxy-5-((S)-tetrahydrofuran-3-yloxy)-benzoate

Prepared by catalytic hydrogenation of benzyl 2-nitro-4-benzyloxy-5-((S)-tetrahydrofuran-3-yloxy)-benzoate in the presence of Raney nickel.

cf. Example IV (1), WO 03/082290

Mass spectrum (ESI⁺): m/z=420 [M+H]⁺

### c) 6-((S)-tetrahydrofuran-3-yloxy)-7-benzyloxy-3H-quinazolin-4-one

Prepared by reacting benzyl 2-amino-4-benzyloxy-5-((S)-tetrahydrofuran-3-yloxy)-benzoate with formamidine acetate.

cf. Example III(1), WO 03/082290

Mass spectrum (ESI⁺): m/z=339 [M+H]⁺

### d) 4-chloro-6-((S)-tetrahydrofuran-3-yloxy)-7-benzyloxy-quinazoline

Prepared by reacting 6-((S)-tetrahydrofuran-3-yloxy)-7-benzyloxy-3H-quinazolin-4-one with thionyl chloride in the presence of N,N-dimethylformamide in acetonitrile at reflux temperature.

cf. Example II(1), WO 03/082290

R_{f} value: 0.85 (silica gel, ethyl acetate/methanol=9:1)

### e) 4-[(3-chloro-4-fluoro-phenyl)amino]-6-((S)-tetrahydrofuran-3-yloxy)-7-benzyloxy-quinazoline-hydrochloride

Prepared by reacting 4-chloro-6-((S)-tetrahydrofuran-3-yloxy)-7-benzyloxy-quinazoline with 3-chloro-4-fluoro-aniline in isopropanol

cf. Example I(1), WO 03/082290

Mass spectrum (ESI⁺): m/z=466, 468 [M+H]⁺

### f) 4-[(3-chloro-4-fluoro-phenyl)amino]-6-((S)-tetrahydrofuran-3-yloxy)-7-hydroxy-quinazoline

Prepared by treating 4-[(3-chloro-4-fluoro-phenyl)amino]-6-((S)-tetrahydrofuran-3-yloxy)-7-benzyloxy-quinazoline with trifluoroacetic acid.

cf. Example 6(1), WO 03/082290

Mass spectrum (ESI⁺): m/z=376, 378 [M+H]⁺

The following may be obtained analogously to Example I:

| Example | Structure |
|---|---|
| I(1) | 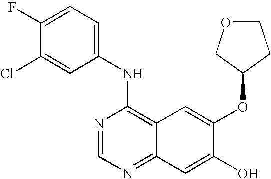 |
| I(2) | 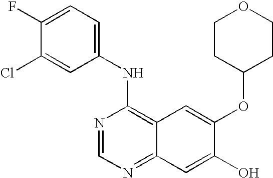 |
| I(3) | 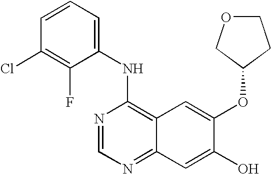 |
| I(4) | 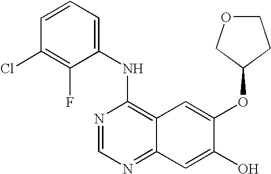 |
| I(5) | 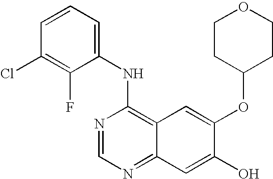 |

### Example II

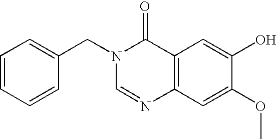

### 3-benzyl-3,4-dihydro-4-oxo-6-hydroxy-7-methoxy-quinazoline

### Method A:

168.5 g 6-hydroxy-7-methoxy-benzo[d][1,3]oxazin-4-one are dissolved in 1200 ml of toluene and 74.7 ml benzylamine are added. The mixture is refluxed for 15 hours and then cooled to ambient temperature. The precipitate is filtered off and washed with tert.-butylmethylether.

Yield 124 g (72% of theory)

### Method B:

200 g 3-benzyl-3,4-dihydro-4-oxo-6-acetyloxy-7-methoxy-quinazoline are suspended in 200 ml of water and 1000 ml of ethanol. 300 ml 10N sodium hydroxide solution are added at ambient temperature and the mixture is heated to 30° C. for 1 hour. After the addition of 172 ml acetic acid and 2000 ml of water the mixture is stirred for 20 hours at ambient temperature. The precipitate is suction filtered, washed with water and acetone and dried at 60° C.

Yield: 172.2 g (98% of theory)

R_{f} value: 0.25 (silica gel, methylene chloride/ethanol=19:1)

Mass spectrum (ESI⁺): m/z=283 [M+H]⁺

### Example III

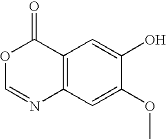

### 6-hydroxy-7-methoxy-benzo[d][1,3]oxazin-4-one

1 g 2-amino-5-hydroxy-4-methoxy-benzoic acid (prepared by reacting methyl 2-nitro-4,5-dimethoxy-benzoate with potassium hydroxide solution to form the potassium salt of 2-nitro-5-hydroxy-4-methoxy-benzoic acid and subsequent catalytic hydrogenation in the presence of palladium on activated charcoal) and 20 ml triethyl orthoformate are heated to 100° C. for 2.5 hours. After cooling to ambient temperature the precipitate is suction filtered and washed with diethyl ether.

Yield: 0.97 g (93% of theory)

R_{f} value: 0.86 (silica gel, methylene chloride/methanol/acetic acid=90:10:1)

Mass spectrum (ESI⁺): m/z=194 [M+H]⁺

### Example IV

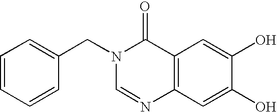

### 3-benzyl-3,4-dihydro-4-oxo-6,7-dihydroxy-quinazoline

200 mg 3-benzyl-3,4-dihydro-4-oxo-6-hydroxy-7-methoxy-quinazoline and 1 g pyridine-hydrochloride are heated to 150° C. for a total of 18 hours. After cooling the reaction mixture is mixed with water and stirred. The solid is separated off by suction filtering, extracted in diethyl ether, suction filtered again and dried.

Yield: 130 mg (68% of theory)

Mass spectrum (ESI⁺): m/z=269 [M+H]⁺

### Example V

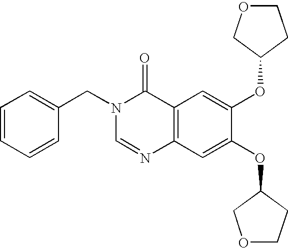

### 3-benzyl-3,4-dihydro-4-oxo-6-((S)-tetrahydrofuran-3-yloxy)-7-((S)-tetrahydrofuran-3-yloxy)-quinazoline

Prepared by reacting 3-benzyl-3,4-dihydro-4-oxo-6,7-dihydroxy-quinazoline with (R)-3-[(4-methylphenyl)sulphonyloxy]-tetrahydrofuran analogously to Example 1

The following may be obtained analogously to Example V:

| Example | Structure |
|---|---|
| V(1) | 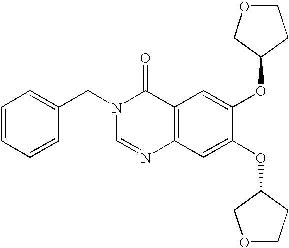 |
| V(2) | 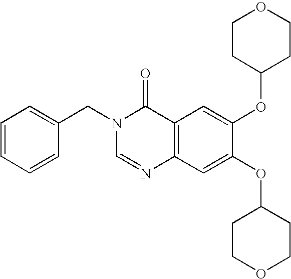 |
| V(3) | 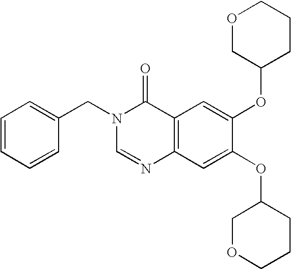 |

### Example VI

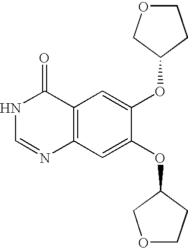

### 3,4-dihydro-4-oxo-6-((S)-tetrahydrofuran-3-yloxy)-7-((S)-tetrahydrofuran-3-yloxy)-quinazoline

Prepared by catalytic hydrogenation of 3-benzyl-3,4-dihydro-4-oxo-6-((S)-tetrahydrofuran-3-yloxy)-7-((S)-tetrahydrofuran-3-yloxy)-quinazoline in glacial acetic acid in the presence of palladium on activated charcoal (10% Pd) at 80° C. and under a hydrogen pressure of 50 psi.

The following may be obtained analogously to Example VI:

| Example | Structure |
|---|---|
| VI(1) | 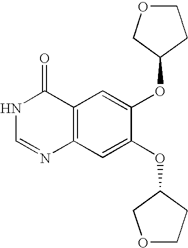 |
| VI(2) | 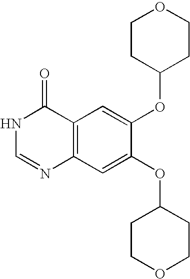 |
| VI(3) | 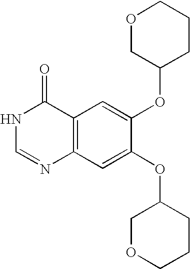 |

### Example VII

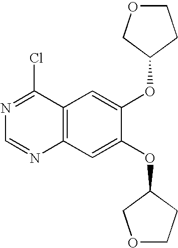

### 4-chloro-6-((S)-tetrahydrofuran-3-yloxy)-7-((S)-tetrahydrofuran-3-yloxy)-quinazoline

Prepared by reacting 3,4-dihydro-4-oxo-6-((S)-tetrahydrofuran-3-yloxy)-7-((S)-tetrahydrofuran-3-yloxy)-quinazoline with thionyl chloride in acetonitrile with the addition of N,N-dimethylformamide.

The following may be obtained analogously to Example VII:

| Example | Structure |
|---|---|
| VII(1) | 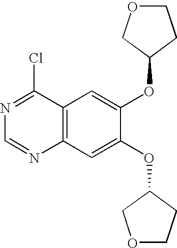 |
| VII(2) | 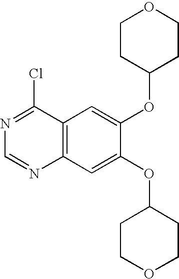 |
| VII(3) | 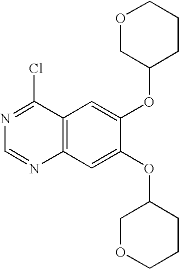 |

### PREPARATION OF THE END COMPOUNDS

### Example 1

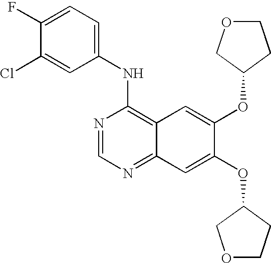

### 4-[(3-chloro-4-fluoro-phenyl)amino]-6-((S)-tetrahydrofuran-3-yloxy)-7-((R)-tetrahydrofuran-3-yloxy)-quinazoline

1 g 4-[(3-chloro-4-fluoro-phenyl)amino]-6-((S)-tetrahydrofuran-3-yloxy)-7-hydroxy-quinazoline, 0.78 g (S)-3-[(4-methylphenyl)sulphonyloxy]-tetrahydrofuran and 0.6 g potassium carbonate are stirred in 10 ml dimethylformamide for 7 hours at 60° C. After the addition of 0.2 g 3-[(4-methylphenyl)sulphonyloxy]-tetrahydrofuran and 0.2 g potassium carbonate the mixture is stirred for another 16 hours at 60° C. After the addition of 0.2 g 3-[(4-methylphenyl)sulphonyloxy]-tetrahydrofuran and 0.2 g potassium carbonate the mixture is again stirred for 4 hours at 60° C. The reaction mixture is cooled, diluted with ethyl acetate and shaken with water and saline solution. The organic phase is dried, evaporated down and the residue is purified by chromatography through a silica gel column with methylene chloride/methanol (98:2 to 90:10). The residue is stirred with diisopropylether and the solid is suction filtered and dried.

Yield: 990 mg (83% of theory)

R_{f} value: 0.44 (silica gel; methylene chloride/methanol=9:1)

Mass spectrum (ESI⁺): m/z=446, 448 [M+H]⁺

The following may be obtained analogously to Example 1:

### (1) 4-[(3-chloro-4-fluoro-phenyl)amino]-6-((S)-tetrahydrofuran-3-yloxy)-7-((S)-tetrahydrofuran-3-yloxy)-quinazoline

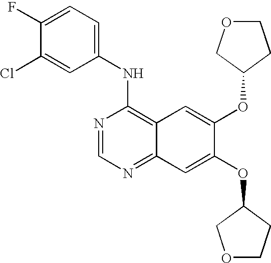

Mass spectrum (ESI⁺): m/z=446, 448 [M+H]⁺

### Example 2

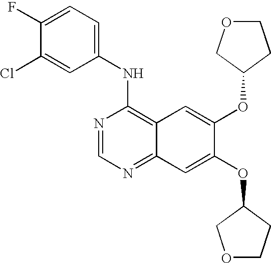

### 4-[(3-chloro-4-fluoro-phenyl)amino]-6-((S)-tetrahydrofuran-3-yloxy)-7-((S)-tetrahydrofuran-3-yloxy)-quinazoline

Prepared by reacting 4-chloro-6-((S)-tetrahydrofuran-3-yloxy)-7-((S)-tetrahydrofuran-3-yloxy)-quinazoline with 3-chloro-4-fluoro-aniline in isopropanol at reflux temperature. The product may be obtained as the hydrochloride or as the free base, depending on how the mixture is worked up.

The following compounds may also be prepared analogously to the foregoing Examples and other methods known from the literature:

| Example | Structure |
|---|---|
| 2(1) | 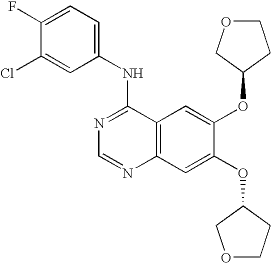 |
| 2(2) | 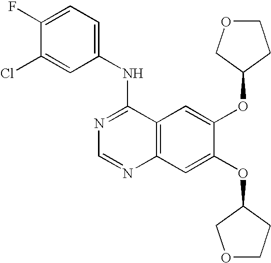 |
| 2(3) | 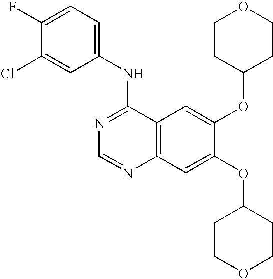 |
| 2(4) | 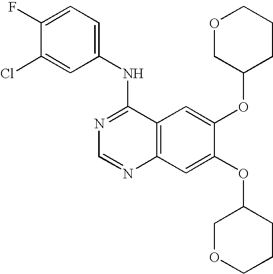 |
| 2(5) | 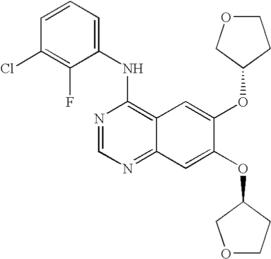 |
| 2(6) | 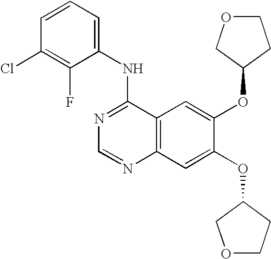 |
| 2(7) | 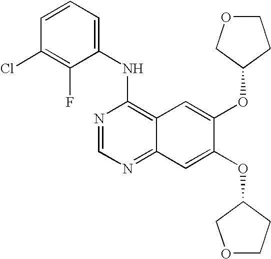 |
| 2(8) | 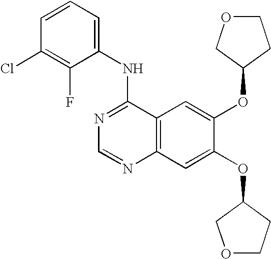 |
| 2(9) | 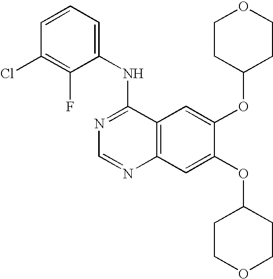 |
| 2(10) | 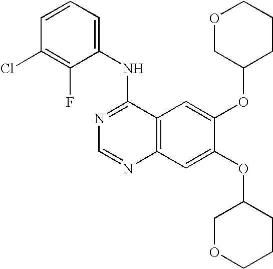 |
| 2(11) | 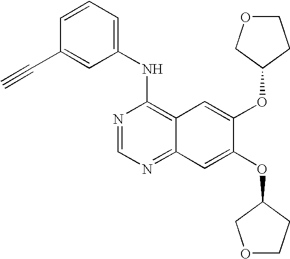 |
| 2(12) | 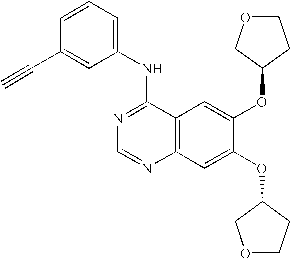 |
| 2(13) | 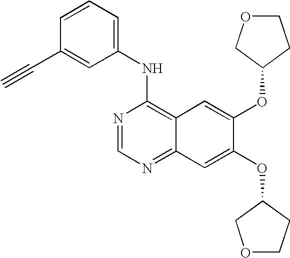 |
| 2(14) | 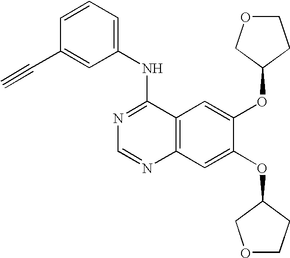 |
| 2(15) | 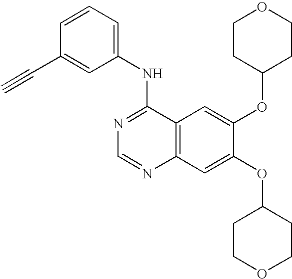 |
| 2(16) | 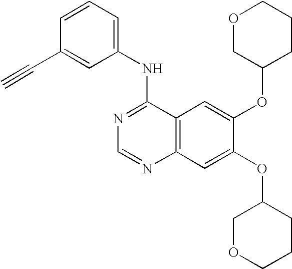 |

### Example 3

### Coated Tablets Containing 75 mg of Active Substance

1 tablet core contains:

| | | |
|---|---|---|
| active substance | 75.0 | mg |
| calcium phosphate | 93.0 | mg |
| corn starch | 35.5 | mg |
| polyvinylpyrrolidone | 10.0 | mg |
| hydroxypropylmethylcellulose | 15.0 | mg |
| magnesium stearate | 1.5 | mg |
| | 230.0 | mg |

### Preparation:

The active substance is mixed with calcium phosphate, corn starch, polyvinylpyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape.
Weight of core: 230 mg
die: 9 mm, convex

The tablet cores thus produced are coated with a film consisting essentially of hydroxypropylmethylcellulose. The finished film-coated tablets are polished with beeswax.
Weight of coated tablet: 245 mg.

### Example 4

### Tablets Containing 100 mg of Active Substance

### Composition:

1 tablet contains:

| | | |
|---|---|---|
| active substance | 100.0 | mg |
| lactose | 80.0 | mg |
| corn starch | 34.0 | mg |
| polyvinylpyrrolidone | 4.0 | mg |
| magnesium stearate | 2.0 | mg |
| | 220.0 | mg |

### Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.
Weight of tablet: 220 mg
Diameter: 10 mm, biplanar, facetted on both sides and notched on one side.

### Example 5

### Tablets Containing 150 mg of Active Substance

### Composition:

1 tablet contains:

| | | |
|---|---|---|
| active substance | 150.0 | mg |
| powdered lactose | 89.0 | mg |
| corn starch | 40.0 | mg |
| colloidal silica | 10.0 | mg |
| polyvinylpyrrolidone | 10.0 | mg |
| magnesium stearate | 1.0 | mg |
| | 300.0 | mg |

### Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.
Weight of tablet: 300 mg
die: 10 mm, flat

### Example 6

### Hard Gelatine Capsules Containing 150 mg of Active Substance

1 capsule contains:

| | | |
|---|---|---|
| active substance | 150.0 | mg |
| corn starch (dried | approx. 180.0 | mg |
| lactose (powdered) | approx. 87.0 | mg |
| magnesium stearate | 3.0 | mg |
| | approx. 420.0 | mg |

### Preparation:

The active substance is mixed with the excipients, passed through a screen with a mesh size of 0.75 mm and homogeneously mixed using a suitable apparatus. The finished mixture is packed into size 1 hard gelatine capsules.
Capsule filling: approx. 320 mg
Capsule shell: size 1 hard gelatine capsule.

### Example 7

### Suppositories Containing 150 mg of Active Substance

1 suppository contains:

| | | |
|---|---|---|
| active substance | 150.0 | mg |
| polyethyleneglycol 1500 | 550.0 | mg |
| polyethyleneglycol 6000 | 460.0 | mg |
| polyoxyethylene sorbitan monostearate | 840.0 | mg |
| | 2,000.0 | mg |

### Preparation:

After the suppository mass has been melted the active substance is homogeneously distributed therein and the melt is poured into chilled moulds.

### Example 8

### Suspension Containing 50 mg of Active Substance

100 ml of suspension contain:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. water | ad 100 | ml |

### Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavouring have been added and dissolved, the suspension is evacuated with stirring to eliminate air.
5 ml of suspension contain 50 mg of active substance.

### Example 9

### Ampoules Containing 10 mg Active Substance

### Composition:

| | | |
|---|---|---|
| active substance | 10.0 | mg |
| 0.01 N hydrochloric acid q.s. | | |
| double-distilled water | ad 2.0 | ml |

### Preparation:

The active substance is dissolved in the necessary amount of 0.01 N HCl, made isotonic with common salt, filtered sterile and transferred into 2 ml ampoules.

### Example 10

### Ampoules Containing 50 mg of Active Substance

### Composition:

| | | |
|---|---|---|
| active substance | 50.0 | mg |
| 0.01 N hydrochloric acid q.s. | | |
| double-distilled water | ad 10.0 | ml |

### Preparation:

The active substance is dissolved in the necessary amount of 0.01 N HCl, made isotonic with common salt, filtered sterile and transferred into 10 ml ampoules.

### Example 11

### Capsules for Powder Inhalation Containing 5 mg of Active Substance

1 capsule contains:

| | |
|---|---|
| active substance | 5.0 mg |
| lactose for inhalation | 15.0 mg |
| | 20.0 mg |

### Preparation:

The active substance is mixed with lactose for inhalation. The mixture is packed into capsules in a capsule-making machine (weight of the empty capsule approx. 50 mg).

weight of capsule: 70.0 mg

size of capsule: 3

### Example 12

### Solution for Inhalation for Hand-Held Nebulisers Containing 2.5 mg Active Substance

1 spray contains:

| | | |
|---|---|---|
| active substance | 2.500 | mg |
| benzalkonium chloride | 0.001 | mg |
| 1N hydrochloric acid q.s. | | |
| ethanol/water (50/50) | ad 15.000 | mg |

### Preparation:

The active substance and benzalkonium chloride are dissolved in ethanol/water (50/50). The pH of the solution is adjusted with 1N hydrochloric acid. The resulting solution is filtered and transferred into suitable containers for use in hand-held nebulisers (cartridges).

Contents of the container: 4.5 g

1. A compound of the formula (I) 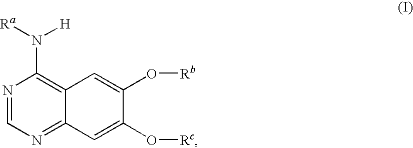
wherein
R^{a} denotes a 3-chloro-2-fluoro-phenyl, 3-chloro-4-fluoro-phenyl or 3-ethynylphenyl group,
R^{b} denotes a tetrahydrofuran-3-yl, tetrahydropyran-3-yl or tetrahydropyran-4-yl- group, and
R^{c} denotes a tetrahydrofuran-3-yl, tetrahydropyran-3-yl or tetrahydropyran-4-yl group,
or a pharmacologically acceptable salt thereof.

2. A compound of the formula (I) according to claim 1,
wherein
R^{b} denotes a tetrahydrofuran-3-yl group, and
R^{c} denotes a tetrahydrofuran-3-yl group,
or a pharmacologically acceptable salt thereof.

3. (canceled)

4. A pharmaceutical composition a compound according to claims 1 or 2 and a pharmaceutically acceptable carrier or diluent.

5. A method for treating COPD which comprises administering to a host suffering from COPD a therapeutically effective amount of a compound according to claim 1 or 2.

6. (canceled)
